(19) United States
(12) Patent Application Publication
(10) Pub. No.: US 2025/0366987 A1
(43) Pub. Date: Dec. 4, 2025

(54) SEALING MEMBERS FOR PROSTHETIC HEART VALVES

(71) Applicant: EDWARDS LIFESCIENCES CORPORATION, Irvine, CA (US)

(72) Inventor: Nikolai Gurovich, Hadera (IL)

(73) Assignee: EDWARDS LIFESCIENCES CORPORATION, Irvine, CA (US)

(21) Appl. No.: 19/303,725

(22) Filed: Aug. 19, 2025

Related U.S. Application Data
(63) Continuation of application No. PCT/US2024/017649, filed on Feb. 28, 2024.
(60) Provisional application No. 63/449,912, filed on Mar. 3, 2023.

(51) Int. Cl. A61F 2/24

(52) U.S. Cl. CPC A61F 2/2418; A61F 2230/0006; A61F 2230/0067; A61F 2250/0069

(57) ABSTRACT

A prosthetic heart valve can include a frame and an outer sealing member disposed around an outer surface of the frame. The frame can include a plurality of rows of angled struts arranged to form a plurality of rows of cells defining openings in the frame, wherein the frame can be radially expandable and compressible between a radially expanded state and a radially compressed state. The outer sealing member can include a base portion extending around the outer surface of the frame and covering the openings of at least one of the rows of cells. The outer sealing member can further include at least one angled portion coupled to the base portion at an inflow end portion thereof and extending radially away from the base portion in a direction from the inflow end to an outflow end portion of the angled portion.

### CROSS REFERENCE TO RELATED APPLICATIONS

This application is a continuation of PCT Application No. PCT/US2024/017649, filed Feb. 28, 2024, which claims the benefit of U.S. Provisional Application No. 63/449,912, filed Mar. 3, 2023. The prior applications are incorporated by reference herein in their entireties.

### FIELD

The present disclosure relates to prosthetic heart valves and relates in particular to sealing members for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic heart valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, prosthetic heart valves comprise a frame or stent and a valvular structure comprising a plurality of leaflets mounted inside the frame. The frame can comprise a plurality of struts that form multiple rows of cells. Prosthetic heart valves can also include a sealing member coupled to the frame to prevent or minimize paravalvular leaks, as well as an inner skirt that can be used to couple the leaflets to the frame and block blood from flowing outwardly through the cells of the frame, typically along the inflow end portion of the frame. Some known prosthetic valves include sealing members that are designed to produce slack that can billow or expand outwardly when the prosthetic valve is in the radially expanded state to enhance the sealing effect of the sealing member against the native annulus. In such prosthetic valves, the inner skirt helps prevent material of the sealing member from protruding inwardly through the cells of the frame and contacting the leaflets, which can interfere with the movement of the leaflets and cause leaflet abrasion over time. However, for prosthetic valves that do not include an inner skirt, such as smaller-diameter prosthetic valves, it may not be possible utilize an outer sealing member that produces slack in the expanded state.

Accordingly, there exists a need for new and improved sealing members, such as can be used for prosthetic valves that do not include an inner skirt.

### SUMMARY

Described herein are prosthetic heart valves, delivery apparatus, and methods for implanting prosthetic heart valves. The disclosed prosthetic heart valves, delivery apparatus, and methods can, for example, provide improved circumferential sealing and paravalvular sealing properties. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, the frame can comprise a plurality of rows of angled struts arranged to form a plurality of rows of cells defining openings in the frame.

In some examples, the frame can be radially expandable and compressible between a radially expanded state and a radially compressed state.

In some examples, at least 90% or more of an inner surface of the frame is exposed and not covered by any material.

In some examples, the valve structure can comprise a plurality of leaflets coupled to the inside of the frame.

In some examples, the prosthetic heart valve can comprise a sealing member with improved circumferential sealing and paravalvular sealing properties.

In some examples, the sealing member can be configured to circumferentially seal the prosthetic heart valve, thereby reducing the flow of blood radially inward through the frame from the external environment surrounding the prosthetic heart valve in the vicinity of the leaflets.

In some examples, the sealing member can be configured to provide paravalvular sealing functionality, thereby reducing paravalvular leakage (PVL) between the frame and the native annulus in which the prosthetic heart valve is implanted.

In some examples, the sealing member can be an outer sealing member disposed on an outer surface of the frame.

In some examples, a prosthetic heart valve can be devoid of an internal sealing member inside of the frame.

In some examples, a prosthetic heart valve can comprise a sealing member configured to reduce contact with the plurality of leaflets of the valve structure.

In some examples, the sealing member can comprise a base portion and at least one angled portion.

In some examples, the base portion can extend around an outer surface of the frame.

In some examples, the base portion can conform to the outer surface of a frame when the frame is in the radially expanded state.

In some examples, the base portion can be tensioned circumferentially when the frame is in the radially expanded state.

In some examples, the base portion can cover openings of at least one rows of cells of the frame.

In some examples, a row of cells covered by the base portion can be at an inflow end of the frame.

In some examples, the angled portion can comprise an outflow end portion disposed opposite an inflow end portion.

In some examples, the angled portion can be coupled to the base portion at an inflow end portion thereof and extend radially away from the base portion in a direction from the inflow end to the outflow end portion of the angled portion.

In some examples, the inflow end portion of the angled portion can be continuously coupled to the base portion along an entirety of the inflow end portion.

In some examples, the outflow end portion of the angled portion can be completely unattached to the base portion.

In some examples, the sealing member can comprise the base portion and a plurality of angled portions coupled to the base portion.

In some examples, at least two angled portions can partially overlap each other in an axial direction of the prosthetic heart valve.

In some examples, none of the angled portions overlap an adjacent angled portion in the axial direction of the prosthetic heart valve.

In some examples, a prosthetic heart valve can comprise a frame. The frame can comprise a plurality of rows of angled struts arranged to form a plurality of rows of cells defining openings in the frame, wherein the frame is radially expandable and compressible between a radially expanded state and a radially compressed state. The prosthetic heart valve can further comprise a valve structure positioned within the frame and configured to regulate the flow of blood through the frame in one direction and an outer sealing member disposed on an outer surface of the frame. The outer sealing member can comprise: a base portion extending around the outer surface of the frame and covering the openings of at least one of the rows of cells; and at least one angled portion coupled to the base portion at an inflow end portion thereof and extending radially away from the base portion in a direction from the inflow end to an outflow end portion of the angled portion.

In some examples, a prosthetic heart valve can comprise: a frame, wherein the frame is radially expandable and compressible between a radially expanded state and a radially compressed state; a valve structure positioned within the frame and configured to regulate the flow of blood through the frame in one direction; and an outer sealing member coupled to an outer surface of the frame. The outer sealing member can comprise: a base portion circumferentially disposed around the outer surface of the frame; and a plurality of annular flap portions positioned along a height of the base portion, wherein each flap portion has an inflow end portion connected to the base portion and an outflow end portion unconnected to the base portion to define a pocket between the flap portion and the base portion that can receive retrograde blood.

In some examples, a prosthetic heart valve can comprise: a frame, wherein the frame is radially expandable and compressible between a radially expanded state and a radially compressed state; a valve structure positioned within the frame and configured to regulate the flow of blood through the frame in one direction; and an outer sealing member disposed on an outer surface of the frame. The outer sealing member can comprise: a base portion coupled to the outer surface of the frame and covering openings in the frame to prevent blood outside of the prosthetic valve from flowing into the frame via the openings and at least one angled portion. The angled portion can comprise: an inflow end portion continuously coupled to the base portion along an entirety of the inflow end portion and an outflow end portion opposite the inflow end portion, wherein the outflow end portion is completely unattached to the base portion.

In some examples, a prosthetic heart valve can comprise one or more of the components recited in Examples 1-55 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of the prosthetic heart valve of FIG. 1.

FIG. 3 is a side view of a frame of a prosthetic heart valve, according to one example.

FIG. 4 is a perspective view of an outer sealing member of a prosthetic heart valve, according to one example.

FIG. 5 is a cross-sectional view of the outer sealing member of FIG. 4.

FIG. 6 is a cross-sectional view of a prosthetic heart valve, wherein a frame of the prosthetic heart valve is in a radially compressed state.

FIG. 7 is a side view of an exemplary delivery apparatus configured to deliver and implant a radially expandable prosthetic heart valve at an implantation site, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

As introduced above, a prosthetic heart valve can include a frame comprising a plurality of interconnected struts and a valve structure comprising a plurality of leaflets mounted inside the frame.

Conventional prosthetic heart valves can include an inner sealing member, which is also referred to herein as an “inner skirt.” The inner sealing member can be configured to provide circumferential sealing of the frame, thereby preventing blood from flowing radially inward through the cells of the frame from the external environment surrounding the prosthetic heart valve in the vicinity of the leaflets.

Conventional prosthetic heart valves can further include an outer sealing member, which is also referred to herein as an “outer skirt.” The outer sealing member can be configured to provide paravalvular sealing, thereby preventing or minimizing blood flow between the prosthetic heart valve and the native annulus in which the prosthetic heart valve is implanted. Known outer sealing members can be configured to billow radially outwards to fill in and occlude gaps between the prosthetic heart valve and the native annulus. As discussed above, in the absence of an inner skirt, such billowing sealing members can also protrude inwardly through the cells of the frame and interfere with the movement of the leaflets and cause unwanted abrasion of the leaflets over time.

Described herein are various prosthetic heart valves with outer sealing members configured to provide both improved circumferential sealing and improved paravalvular sealing. The outer sealing members described herein can beneficially have both the circumferential sealing functionality of a conventional inner skirt and the paravalvular sealing functionality of a conventional outer sealing member.

Thus, in some examples, the outer sealing members described herein can be configured to beneficially allow for the elimination of inner sealing members. Eliminating inner sealing members from prosthetic heart valve designs can beneficially improve long term durability of leaflets by avoiding contact between the leaflets and an inner skirt. Further, omitting an inner sealing member can facilitate assembly of a prosthetic valve by eliminating the stitching required to assemble the leaflets to the inner sealing member and the inner sealing member to the frame, thereby greatly reducing assembly time and manufacturing costs. However, it should be noted that the outer sealing members discloses herein can be used with prosthetic valves that do have inner sealing members.

### Examples of the Disclosed Technology

Prosthetic heart valves disclosed herein can be radially compressible and expandable between a radially compressed state and a radially expanded state. Thus, the prosthetic heart valves can be crimped on or retained by an implant delivery apparatus in the radially compressed state during delivery, and then expanded to the radially expanded state once the prosthetic heart valve reaches the implantation site. It is understood that the prosthetic heart valves disclosed herein may be used with a variety of implant delivery apparatuses and can be implanted via various delivery procedures, examples of which will be discussed in more detail later.

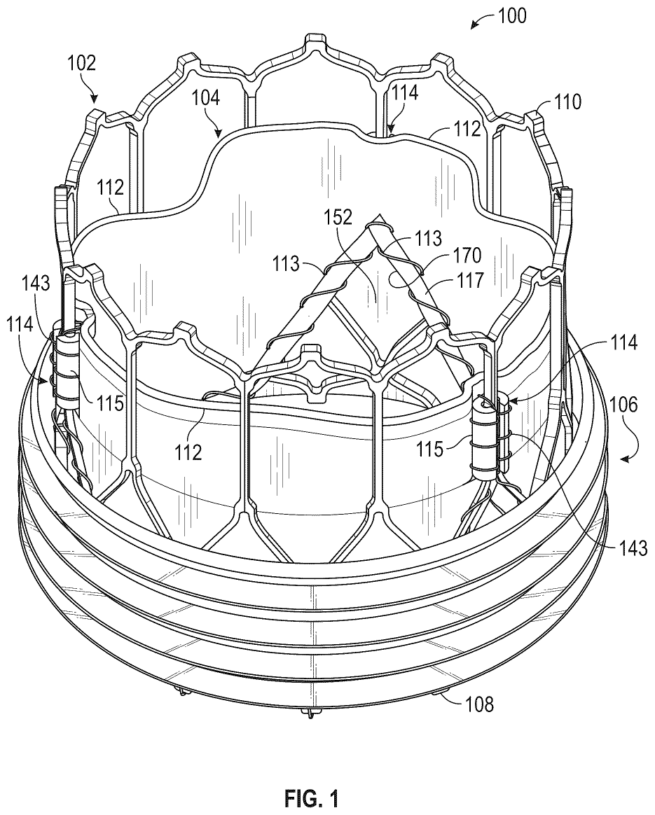

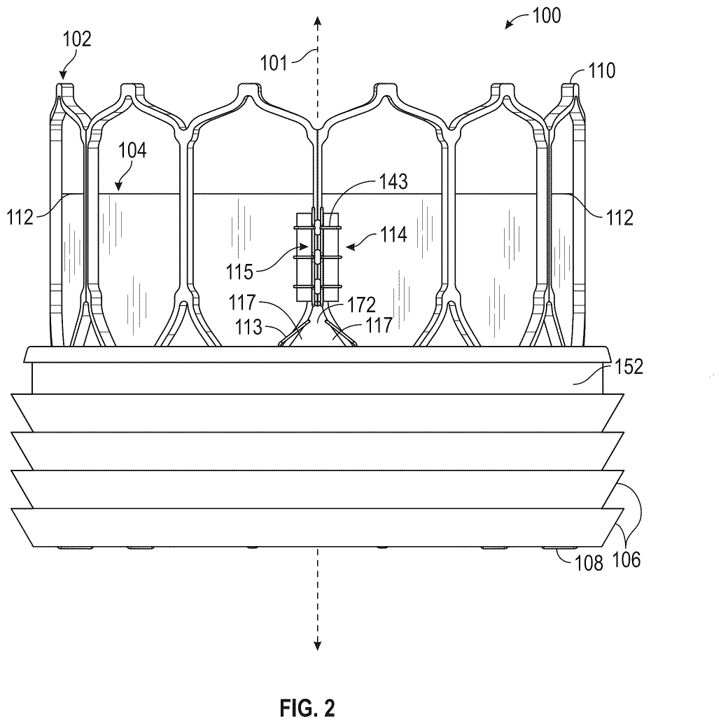

FIGS. 1-2 show a prosthetic heart valve 100 (which is also referred to herein as a “prosthetic valve”), according to one example. Any of the prosthetic heart valves disclosed herein are adapted to be implanted in the native aortic annulus, although in other examples they can be adapted to be implanted in the other native annuluses of the heart (the pulmonary, mitral, and tricuspid valves). The disclosed prosthetic heart valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic heart valves also can be implanted within a previously implanted prosthetic heart valve (which can be a prosthetic surgical valve or a prosthetic transcatheter heart valve) in a valve-in-valve procedure.

In some examples, the disclosed prosthetic heart valves can be implanted within a docking or anchoring device that is implanted within a native heart valve or a vessel. For example, in one example, the disclosed prosthetic heart valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic heart valves can be implanted within a docking device implanted within or at the native mitral valve, such as disclosed in PCT Publication No. WO2020/247907, which is incorporated by reference herein. In another example, the disclosed prosthetic heart valves can be implanted within a docking device implanted within the superior or inferior vena cava for replacing the function of a diseased tricuspid valve, such as disclosed in U.S. Publication No. 2019/0000615, which is incorporated by reference herein.

The prosthetic heart valve 100 can comprise a frame 102 and a valve structure 104 disposed inside the frame 102. In some examples, the prosthetic heart valve 100 can further comprise an outer sealing member 106 disposed on an outer surface of the frame 102.

The valve structure 104 can be configured to regulate the flow of blood through the frame 102 in one direction. The valve structure 104 can comprise a plurality of leaflets 112 (such as three leaflets, as shown in FIG. 1), collectively forming a leaflet structure. The leaflet structure can be arranged to collapse in a tricuspid arrangement. The leaflets 112 can be secured to one another or to the frame 102 at their adjacent sides (for example, commissure tabs) to form commissures 114 of the valve structure 104. For example, each leaflet can comprise opposing commissure tabs 115 disposed on opposite sides of the leaflet 112 and a cusp edge portion 117 extending between the opposing commissure tabs 115. In some examples, the cusp edge portion 117 of the leaflets 112 can have an undulating, curved scallop shape.

The cusp edge portion 117 of each leaflet 112 can be secured to the frame 102 via one or more fasteners (for example, sutures). In some examples, the cusp edge portion 117 of each leaflet 112 can be secured directly to the struts of the frame 102. For example, as shown in FIGS. 1-2, the cusp edge portions 117 of the leaflets 112 can be secured via sutures 113 to struts that generally follow the contour of the cusp edge portions 117 of the leaflets 112. Additional methods for securing the leaflets 112 to the frame 102 are disclosed in PCT Application No. PCT/US2022/049666 (published as PCT Publication No. WO 2023/086548), filed Nov. 11, 2022, which is incorporated by reference herein.

In some examples, the leaflets 112 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

As shown in FIG. 1, the prosthetic heart valve 100 can be devoid of an inner sealing member or inner skirt disposed on an inner surface of the frame 102. Thus, in such examples, the prosthetic valve 100 can be devoid of any material on the inner surface of the frame 102, except for the cusp edge portions 117, the commissure tabs 115, and any components that may be positioned along the cusp edge portions 117 and/or the commissure tabs 115 to attach those components of the leaflets to the frame 102 (for example, sutures and/or relatively small reinforcing members used to secure the cusp edge portions 117 and/or the commissure tabs 115 to the frame). In some examples, the prosthetic valve 100 is devoid of any material on the inner surface of the frame 102 that blocks or occludes any of the cells 118 of the frame 102. In some examples, at least 90% or more of the inner surface of the frame 102 is exposed and not covered by any material; in some examples, at least 95% or more of the inner surface of the frame 102 is exposed and not covered by any material; and in some examples, at least 98% or more of the inner surface of the frame 102 is exposed and not covered by any material.

In some known prosthetic valves that include an inner sealing member disposed between the inner surface of the frame and the valve structure, the moveable portions of the leaflets may contact the inner sealing member, potentially causing abrasion of the leaflets. Advantageously, by omitting the inner sealing member, the prosthetic heart valve 100 can have increased durability and longevity.

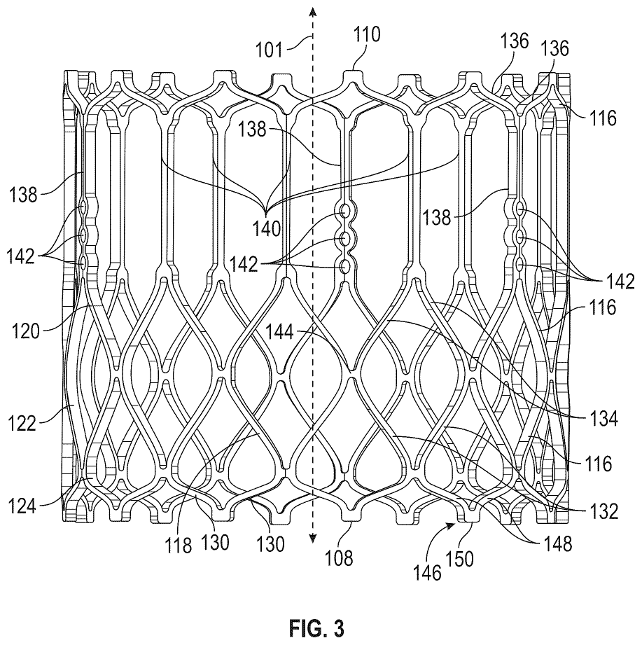

FIG. 3 shows the frame 102 of the prosthetic heart valve 100, according to one example. The frame 102 can be radially compressible and expandable between a radially compressed state and a radially expanded state. The frame 102 can be annular, in that it defines in the radially expanded state an annulus through which blood can flow.

The frame 102 can comprise a plurality of interconnected struts 116 which form multiple rows of cells 118 between an inflow end 108 and an outflow end 110 of the frame 102. In some examples, the frame 102 can comprise first, second, and third rows of cells 120, 122, 124, respectively. The first row of cells 120 can be disposed at the outflow end 110 of the frame 102. The first row of cells 120 can comprise cells 118 that are elongated in an axial direction (relative to a central longitudinal axis 101 of the frame 102) as compared to cells 118 in the second and third rows of cells 122, 124. The third row of cells 124 can be disposed at the inflow end 108 of the frame 102. The second row of cells 122 can be disposed between the first row of cells 120 and the third row of cells 124.

In some examples, such as the example shown in FIG. 3, each row of cells can comprise twelve cells 118. Thus, in such examples, the frame 102 can be referred to as a “twelve-cell frame.”

In some examples, the frame 102 can comprise more than three rows of cells 118 (for example, four or five rows), less than three rows of cells (one or two rows of cells), and/or more or less than twelve cells per row. In some examples, the cells 118 in the first row of cells 120 may not be axially elongated compared to cells 118 in the remaining rows of cells (the second row of cells 122 and the third row or cells 124).

The interconnected struts 116 can include a plurality of angled struts 130, 132, 134, 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of first axially extending struts 138 (or first axial struts 138) and a plurality of second axially extending struts 140 (or second axial struts 140). Each of the first axial struts 138 (which can also be referred to as commissure support struts) can define one or more commissure apertures or eyelets 142, which are adapted to receive sutures 143 (FIG. 1) for securing the commissure tabs 115 of a pair of adjacent leaflets 112 to the frame 102. In some examples, each of the first axial struts 138 can include three commissure apertures 142. In some examples, the commissure apertures 142 can be circular openings in the aperture struts 138. In some examples, the portions of the first axial struts 138 that include the commissure apertures 142 can be referred to herein as commissure features, wherein each commissure feature can be configured to be secured to a pair of commissure tabs 115 of a pair of adjacent leaflets 112.

One or more (for example, three, as shown in FIG. 3) second axial struts 140 can be positioned between, in the circumferential direction, two first axial struts 138.

Each second axial strut 140 and each first axial strut 138 can extend from a location defined by the convergence of the lower ends (for example, ends arranged inward of and farthest away from the outflow end 110) of two angled struts 136 (which can also be referred to as an upper strut junction or upper elongated strut junction) to another location defined by the convergence of the upper ends (for example, ends arranged closer to the outflow end 110) of two angled struts 134 (which can also be referred to as a lower strut junction or lower elongate strut junction). Each second axial strut 140 and each first axial strut 138 can form an axial side of two adjacent cells 118 of the first row of cells 120.

The interconnected struts 116 can also comprise horizontal struts 144 that extend between adjacent cells 118 of a row of cells of the frame 102 (FIGS. 2 and 3). The horizontal struts 144 can extend in a circumferential direction and also be referred to as circumferentially extending struts 144. The horizontal struts 144 can connect angled struts of two adjacent rows of angled struts of the frame 102 to one another. For example, each horizontal strut 144 can connect to two angled struts of one row of struts (for example, struts 134 shown in FIG. 3) and two angled struts in another, adjacent row of struts (for example, struts 132 shown in FIG. 3). As a result, an angled strut 134 extending between the first axial strut 138 and the horizontal strut 144 and an angled strut 134 extending between the horizontal strut 144 and another horizontal strut 144 disposed adjacent to the inflow end 108 of the frame 102 can be aligned along an angled line that can follow a scallop line of the leaflets (when the leaflets are attached to the frame 102). Thus, the horizontal struts 144 can allow the angled struts to follow a shape that more closely matches a shape of the scallop line of the leaflets when the frame 102 is in the radially expanded configuration (as shown in FIGS. 1, 2, and 3). Additionally, the horizontal struts 144 can serve as spacers that can maintain a specified gap between the angled struts when the frame 102 is in the radially state configuration, thereby reducing a risk of pinching the leaflets between the struts in the radially state configuration.

The frame 102 can further comprise a plurality of apex regions 146 formed at the inflow end 108 and the outflow end 110, wherein each apex region 146 can form a junction between two angled struts 130 at the inflow end 108 or two angled struts 136 at the outflow end 110. As such, the apex regions 146 are spaced apart from one another, in a circumferential direction at the inflow end 108 and the outflow end 110.

In some examples, as shown in FIG. 3, each apex region 146 can have side portions 148 that curve or bend axially outward from the angled strut 130, 136 to which it is connected and an end portion 150 that extends between the two side portions 148 of the apex region 146. The side portions 148 can extend in a direction that is parallel to the central longitudinal axis 101. The end portion 150 can be relatively flat and include a surface that is disposed normal to the central longitudinal axis 101. Each apex region 146 can have two bends at its end portion 150 and two bends at the side portions 148 (for example, one at the junction between each side portion 148 and angled strut 130, 136). In this way, the apex regions 146 can be U-shaped. However, in some examples, the apex regions 146 can have another shape such as a more curved and longer apex region with a reduced height in the axial direction, such as disclosed in PCT Publication No. WO 2022/226147, filed Apr. 21, 2022, which is incorporated by reference herein.

The frame 102 can be made of any of various suitable plastically-expandable materials (for example, stainless steel) or self-expanding materials (for example, Nitinol) as known in the art. When constructed of a plastically-expandable material, the frame 102 (and thus the prosthetic heart valve 100) can be crimped to the radially compressed state on a delivery catheter and then expanded inside a patient by an inflatable balloon or equivalent expansion mechanism. When constructed of a self-expandable material, the frame 102 (and thus the prosthetic heart valve 100) can be crimped to the radially compressed state and restrained in the compressed state by insertion into a sheath or equivalent mechanism of a delivery catheter. Once inside the body, the valve can be advanced from the delivery sheath, which allows the valve to expand to its functional size in the radially expanded state.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

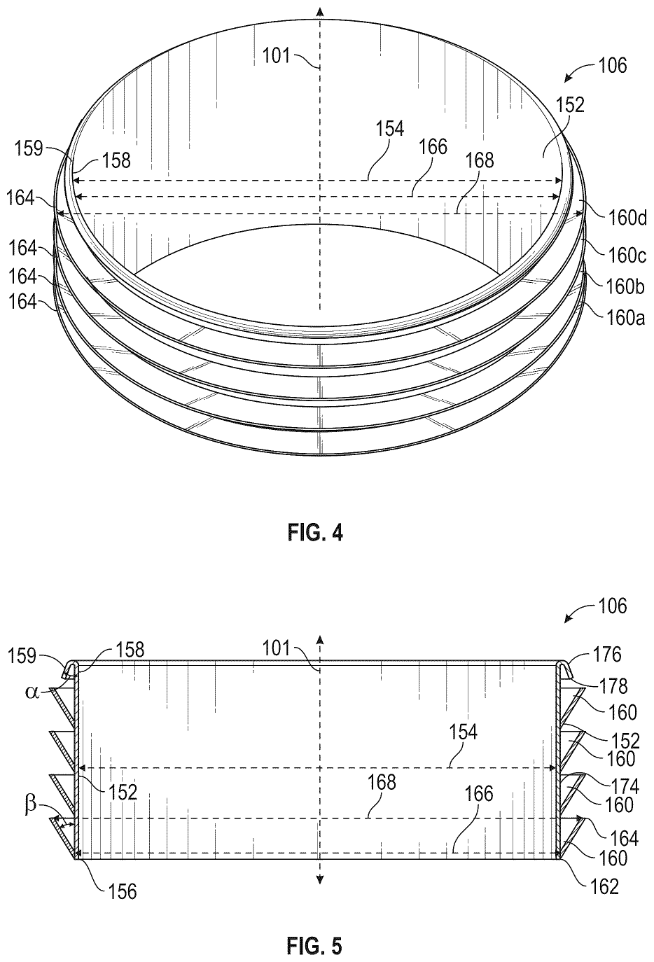

FIGS. 4-5 show the outer sealing member 106 of the prosthetic heart valve 100, according to one example. The outer sealing member 106 can be configured to provide for both circumferential sealing, which blocks the flow of blood inwardly through the cells 118 of the frame 102 from the external environment surrounding the prosthetic heart valve in the vicinity of the leaflets 112, and paravalvular sealing, which reduces paravalvular leakage (PVL) between the prosthetic heart valve 100 and the native annulus in which the prosthetic heart valve 100 is implanted.

The outer sealing member 106 can comprise a base portion 152 (which can also be referred to as an inner wall of the sealing member) disposed on and extending around an outer surface of the frame 102. The base portion 152 can be configured to circumferentially seal the prosthetic heart valve 100 and block the flow of blood inwardly through the cells 118 of the frame 102 from the external environment surrounding the prosthetic heart valve in the vicinity of the leaflets 112. In some examples, the base portion 152 desirably is sized and shaped relative to the frame such that when the prosthetic valve 100 is in its radially expanded state, the base portion 152 conforms to the outer surface of the frame 102. In some examples, the base portion 152 can be sized and shaped such that when the prosthetic valve 100 is in its radially expanded state, the base portion fits snugly against the outer surface of the frame 102 with little or no material slack in the base portion 152.

The base portion 152 can be configured to form an annular shape when disposed on the outer surface of the frame 102, such that at least a portion of the frame 102 is disposed within the annulus of the base portion 152. Thus, the base portion 152 can extend along the entirety of the circumference of the frame 102 when the base portion 152 is disposed on the outer surface of the frame 102.

The annular base portion 152 can define a base portion diameter 154 that is perpendicular to the axial direction defined by the central longitudinal axis 101 of the prosthetic heart valve 100 and the outer sealing member 106. In some examples, in which the base portion 152 conforms to the outer surface of the frame 102, the base portion diameter 154 can be equal to the outer diameter of the frame 102 in its radially expanded state. Ensuring that the base portion 152 conforms to the outer surface of the frame 102, for example by minimizing the difference between the base portion diameter 154 and the expanded diameter of the frame 102, can beneficially avoid or minimize slack in the outer sealing member 106, thereby ensuring that the base portion 152 does not collapse or protrude radially inwardly through the cells 118 of the frame 102 and contact the leaflets 112 during operation of the prosthetic heart valve 100.

In some examples, the base portion 152 can be sized and shaped such that when the prosthetic heart valve 100 is in its radially expanded state, the base portion 152 can be tensioned or stretched in a circumferential direction so as to conform to the outer surface of the frame 102 in a tight-fitting manner. Tensioning the base portion 152 circumferentially can beneficially help reduce slack in the base portion 152, thereby preventing portions of the base portion 152 from collapsing or protruding radially inward through the cells 118 of the frame 102 and contacting the one or more of the plurality of leaflets 112.

In some examples, the base portion 152 can better conform to the outer surface of the frame 102 if the base portion 152 has the same shape as the frame 102. For example, if the frame 102 is cylindrical, as shown best in FIGS. 1-2, the base portion 152 can better conform to the outer surface of the frame 102 if the base portion 152 also has a cylindrical shape. In some examples, the frame 102 can have a non-cylindrical shape in which the diameter of the frame 102 can vary along its length, such as a tapered shape (in which the diameter of the inflow end 108 is greater than the diameter of the outflow end 110, or vice versa), or an hour-glass shape. In such as case, the base portion 152 can have the same shape as the portion of the frame 102 covered by the base portion 152.

The base portion 152 can comprise an inflow end portion 156 and an outflow end portion 158. The inflow end portion 156 can be disposed towards the inflow end 108 of the frame 102, while the outflow end portion 158 can be disposed towards the outflow end 110 of the frame 102. Thus, the inflow end portion 156 and the outflow end portion 158 can be opposing end portions of the base portion 152.

In some examples, the base portion 152 can be configured to cover the entire outer surface of the frame 102. Thus, in such examples, the inflow end portion 156 of the base portion 152 can be coterminous in the axial direction with the inflow end 108 of the frame 102 and the outflow end portion 158 of the base portion 152 can be coterminous in the axial direction with the outflow end 110 of the frame 102.

In some examples, the base portion 152 can be configured to cover only a portion of the frame. As best shown in FIG. 2, the inflow end portion 156 of the base portion 152 can be coterminous in the axial direction with the inflow end 108 of the frame 102, but the outflow end portion 158 of the base portion 152 does not extend in the axial direction to the outflow end 110 of the frame 102. Instead, the outflow end portion 158 can extend to a level of the leaflets 112 where the downstream ends of cusp edge portions 117 of adjacent leaflets intersect each other just upstream of the commissures 114. In this manner, the base portion 152 can cover the openings in the frame 102 that are located at gaps 170 between adjacent cusp edge portions 117 of adjacent leaflets, thereby blocking the inward flow of blood through the frame 102 and the gaps 170 between the leaflets 112 (see FIG. 1). This blocks the flow of retrograde blood through the frame 102 in the radial direction to the upstream side of the leaflets 112.

In some examples, the outflow end portion 158 of the base portion can extend to sub-commissure regions 172 of the leaflets, which are portions of the valve structure 104 where adjacent edges of adjacent leaflets 112 are secured to each other immediately upstream of the commissures 114. The sub-commissure regions 172 can be formed by securing (such as with sutures) adjacent cusp edge portions 117 of adjacent leaflets 112 to each other. The portion of the frame 102 downstream of the sub-commissure regions 172 can be uncovered by the outer sealing member 106 (including the base portion 152) to maximize the flow of blood to the coronary arteries and access to the coronary arteries (when the prosthetic heart valve 100 is implanted within the native aortic valve). Thus, in some examples, the base portion 152 can be disposed closer to the inflow end 108 than the outflow end 110 of the frame 102. Such a configuration of the base portion 152 can minimize the size of the outer sealing member 106 while still circumferentially sealing the gaps 170 between the cusp edge portions 117 of the leaflets 112.

In some examples, the base portion 152 comprises a main body 174 and the outflow end portion 158 can be folded radially outwards and downwards (in an upstream direction) relative to the main body 174 to form a folded portion or flap 176 defining a terminal edge 178 of the base portion 152. To the extent the base portion 152 is formed from a woven fabric, the terminal edge 178 can be a molten edge configured to prevent fraying of the fabric. Forming the folded portion 176 can beneficially prevent contact between the leaflets 112 and the molten edge of the outflow end portion 158, further protecting the leaflets 112 against abrasion. In some examples, the folded portion 176 can be disposed radially outwards of the main body 174. As shown best in FIG. 5, the main body 174 and the folded portion 176 can form an angle (a) of approximately 20 degrees (for example, ±5 degrees) when the prosthetic heart valve 100 is in the radially expanded state. In some examples, the folded portion can be folded flush against the main body 174.

In some examples, the inflow end portion 156 of the base portion 152 can comprise a molten edge to prevent the inflow end portion 156 from fraying. Thus, the inflow end portion 156 can additionally or alternatively be folded in the radially outward and upward (in an upstream direction), for example, to further reduce potential contact between the inflow end portion 156 and the leaflets 112 when the prosthetic heart valve 100 is in the radially expanded state.

The base portion 152 can be coupled to or secured to the frame 102 using various techniques and/or mechanisms. In some examples, the base portion 152 can be coupled to the frame 102 using mechanical fasteners, sutures, adhesives, or other methods known in the art. In some examples, the inflow end portion 156 can be secured with sutures to selected struts 130 of the first row of angled struts and the outflow end portion 158 can be secured with sutures to selected struts 134 of the third row of angled struts. Additional methods of coupling sealing members to frames are disclosed in U.S. Pat. No. 9,393,110, which is incorporated by reference herein.

The outer sealing member 106 can further comprise one or more angled portions 160, which can also be referred to herein as “flap portions” or “tapered portions.” The angled portions 160 can be configured to provide the outer sealing member 106 with paravalvular sealing functionality by occluding gaps between the prosthetic heart valve 100 and the native annulus during the operation of the prosthetic heart valve 100.

The angled portions 160 can be disposed on an outer surface of the base portion 152. In some examples, each angled portion 160 can be configured to extend completely around the base portion 152 in a circumferential direction, thereby forming an annular shape when disposed on the outer surface of the base portion 152 such that at least a portion of the frame 102 and the base portion 152 are disposed within the annulus of the angled portion 160.

Each angled portion 160 can comprise an inflow end portion 162 disposed towards the inflow end 108 of the frame 102 and an outflow end portion 164 disposed towards the outflow end 110 of the frame 102. In some examples, the inflow end portion 162 can be continuously coupled to the base portion 152 in the circumferential direction such that blood cannot flow between the inflow end portion 162 of the angled portion 160 and the base portion 152. The inflow end portion 162 of the angled portion 160 can be secured or coupled to the base portion 152 using mechanical fasteners, sutures, adhesives, or ultrasonic welds.

The inflow end portion 162 can define an inflow end portion diameter 166. When the inflow end portion 162 is coupled to the base portion 152, the inflow end portion diameter 166 can be equal to the base portion diameter 154.

The outflow end portion 164 can define an outflow end portion diameter 168 that can be greater than the base portion diameter 154 and the inflow end portion diameter 166. Thus, when the outflow end portion diameter 168 is greater than the inflow end portion diameter 166, each angled portion 160 can assume a frustoconical shape, in which the inflow end portion 162 can correspond to the narrower end of the frustoconical shape and the outflow end portion 164 can correspond to the wider end of the frustoconical shape. Furthermore, when the angled portion 160 assumes the frustoconical shape, the outflow end portion 164 can be spaced radially away from the base portion 152.

When the angled portion 160 assumes the frustoconical shape, the angled portion 160 tapers from the outflow end portion 164 to the inflow end portion 166 in the upstream direction at an angle (β). The angle (β), which can also be defined as the angle formed between the base portion 152 and the angled portion 160, can be any angle less than 90 degrees. In some examples, the angle (β) is approximately 40 degrees (for example, ±5 degrees).

When each angled portion 160 assumes the frustoconical shape, the outflow end portion 164 of the angled portion 160 can be unattached to the base portion 152. In some examples, each angled portion 160 can be completely unattached to the base portion 152 along an entirety of the outflow end portion 164 of the angled portion 160, thus rendering the outflow end portion 164 free to move radially away or towards the base portion 152. In some examples, no part of the outflow end portion 164 contacts the base portion 152 when the angled portion 160 assumes the frustoconical shape.

The base portion 152 and the angled portions 160 can define pockets when the angled portions 160 assumes their frustoconical shape. The base portion 152 and the outflow end portion 164 of each angled portion 160, which can be uncoupled to and spaced radially away from the base portion 152, can define a pocket opening facing the outflow end 110 of the frame 102. Once implanted in the annulus of a native heart valve (for example, the native aortic valve), the angled portions 160 can expand outwardly and contact the tissue of the native annulus. Retrograde blood can flow into and accumulate in the pockets, effectively pushing the angled portions 160 outwardly against the annulus such that the angled portions can better conform to the irregular surface of the annulus, thereby establishing a seal with the native annulus to prevent or minimize paravalvular leakage around the prosthetic heart valve 100. In some examples, the pockets and the pocket openings can extend around the entire circumference of the frame 102.

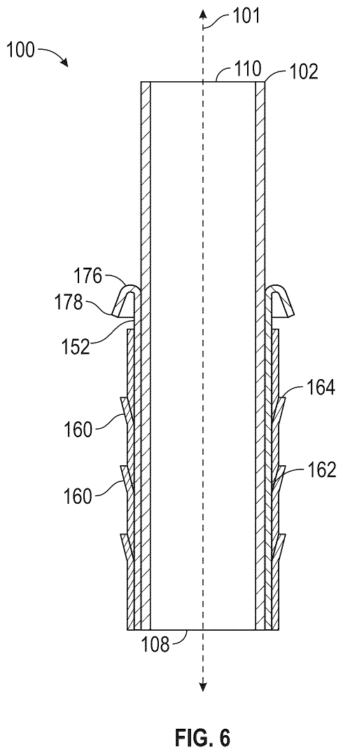

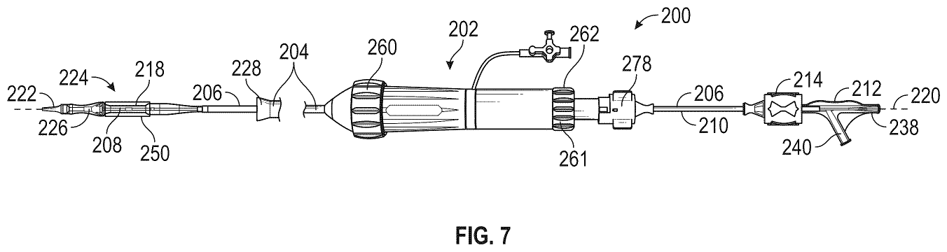

In some examples, the outer sealing member 106 can assume a first configuration when the frame 102 is in the radially expanded state (FIG. 5) and can assume a second configuration when the frame 102 is in the radially compressed state (FIG. 6). In the first configuration, the angled portions 160 can assume their frustoconical shape. However, in the second configuration, the angled portions 160 can assume a flattened configuration, in which the angled portions 160 are flattened against an outer surface of the base portion 152 such that the angled portions 160 are oriented substantially parallel to the base portion 152, but still unattached to the base portion 152 at the outflow end portion 164. Allowing the outer sealing member 106 to assume the second configuration, in which the angled portions 160 do not extend radially outward, can beneficially reduce the compressed diameter of the prosthetic heart valve 100 when the prosthetic heart valve 100 is crimped to a delivery apparatus (for example, the delivery apparatus of FIG. 7) and delivered to the implantation site.

In some examples, the outer sealing member 106 can be shape set such that the angled portions 160 assumes their frustoconical shape when the prosthetic heart valve 100 is deployed to the radially expanded configuration. The angled portions 160 can be shape set in an original state to assume the frustoconical shape. In some examples, when the prosthetic heart valve 100 is radially compressed for delivery, the angled portions 160 can be deformed to the second (compressed) configuration as shown in FIG. 6 can be retained in the second configuration with a restraint placed around the outer sealing member 106. For example, the restraint can be a sheath sized to be placed over the outer sealing member 106 or one or more lasso members (for example, formed from sutures) encircling respective angled portions 160. When the prosthetic heart valve 100 is positioned at the intended deployment site (for example, within a native heart valve annulus), the restraint can be removed, thereby allowing the angled portions 160 to self-expand to their expanded, frustoconical shape of the first configuration. In some examples, each of the angled portions 160 can include one or more expansion assist elements, such as in the form of shape-memory metal struts or wires (for example, Nitinol struts or wires), that assist in causing the angled portions 160 to expand from the compressed configuration (FIG. 6) to the expanded configuration (FIG. 5).

In some examples, the angled portions 160 are not shape set and do not expand under their own resiliency. Instead, the angled portions 160 can move from the compressed configuration to the expanded configuration with the aid of retrograde blood flowing into the pockets defined between the angled portions 160 and the base portion 152.

In some examples, the outer sealing member 106 can comprise a plurality of angled portions 160 positioned along the height of the base portion 152, as shown in the figures. In some examples, the outer sealing member 106 can comprise four angled portions 160 (such as first, second, third, and fourth angled portions 160a, 160b, 160c, 160d shown in FIG. 4), but the outer sealing member 106 can alternatively comprise one, two, three, five, six, seven, or more angled portions 160.

In examples where the outer sealing member 106 comprises a plurality of angled portions 160, at least one of the plurality of angled portions 160 can at least partially overlap an adjacent angled portion 160 in the axial direction of the prosthetic heart valve 100 when the prosthetic heart valve 100 is in the radially expanded state. In other words, when two angled portions 160 overlap each other, at least the outflow end portion 164 of a first angled portion 160 overlaps the inflow end portion 162 of a second, adjacent angled portion 160, downstream of the first angled portion 160, in the axial direction.

In examples where the outer sealing member 106 comprises a plurality of angled portions 160, at least two of the angled portions 160 can be non-overlapping in the axial direction when the prosthetic heart valve 100 is in the radially expanded state. When two adjacent angled portions 160 do not overlap, an outflow end portion 164 of a first angled portion 160 can be spaced or offset in the axial direction from the inflow end portion 162 of a second, adjacent angled portion 160 that is downstream of the first angled portion 160. In some examples, none of the plurality of angled portions 160 overlap an adjacent angled portion 160 in the axial direction when the prosthetic heart valve 100 is in the radially expanded state.

In some examples, as shown in FIG. 5, the height of each angled portion 160 can be the same. In other examples, the angled portions 160 can have different heights.

As shown in FIG. 5, in some examples, angled portions 160 can be positioned along the entire height or substantially the entire height of the base portion 152. In other examples, sections of the base portion 152 can be without any angled portions 160. For example, as shown in FIG. 2, an outflow end region of the base portion 152 can be devoid of an angled portion. In some examples, an inflow end region of the base portion 152 can be devoid of an angled portion.

Moreover, in some examples, the sealing member 106 can have exactly one angled portion 160. For example, the sealing member 106 can have any one of angled portions 160a, 160b, 160c, 160d, while the others can be omitted. When a single angled portion 160 is provided, the height of the angled portion 160 can be substantially less than the height of the base portion. Alternatively, the height of the angled portion can be the same or substantially the same as the overall height of the base portion 152. For example, the single angled portion 160 can have an inflow end portion 162 secured to the inflow end portion 156 of the base portion 152 and the outflow end portion 164 can be at the same level of the outflow end portion 158 of the base portion 152 in the axial direction. In this manner, the single angled portion 160 extends over and covers the entire or substantially the entire extent of the base portion 152.

The outer sealing member 106 can be wholly (for example, both the base portion 152 and the angled portion 160) or partly formed of any suitable biological material, synthetic material (for example, any of various polymers), or combinations thereof. In some examples, the outer sealing member 106 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer sealing member 106 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer sealing member 106 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the outer sealing member 106 can comprise a sponge material or foam, such as polyurethane foam. In some examples, the outer sealing member 106 can comprise natural tissue, such as pericardium (for example, bovine pericardium, porcine pericardium, equine pericardium, or pericardium from other sources).

In some examples, the base portion 152 and the angled portions 160 can be separately formed and subsequently connected to each other, such as with sutures, mechanical fasteners, an adhesive, by welding, or other connection means. In other examples, the base portion 152 and the angled portions 160 can be integrally formed with each other.

The base portion 152 and the angled portions 160 can be formed from the same material or different materials. For example, in some examples, the base portion 152 can be formed from a first material and the angled portions 160 can be formed from a second material. The first material can be non-porous or substantially non-porous to prevent blood from flowing radially into the frame 102 through the cells 118 and/or can have a surface texture selected to minimize tissue ingrowth and a thrombogenic response, while the second material can be selected to encourage tissue ingrowth. For example, the first material can comprise a plain woven fabric (for example, a plain woven PET fabric), a non-textile polymeric film (for example, a TPU film), or natural tissue, while the second material can comprise a fabric having a plush nap or pile to promote tissue ingrowth.

When the frame 102 is radially compressed from the radially expanded state (FIGS. 1-3) to the radially compressed state (FIG. 6), the frame 102 decreases in diameter and increases in length. Conversely, when the frame 102 is radially expanded from the radially compressed state to the radially expanded state, the frame 102 increases in diameter and decreases in length. If the inflow end portion 156 and the outflow end portion 158 of the base portion 152 are securely connected to adjacent rows of struts (such as with sutures), radially compression of the frame can produce elongation or stretching of the base portion 152 in the axial direction. In some examples, the material and/or the construction of the base portion 152 can be selected to allow the base portion 152 to expand or stretch axially when the prosthetic heart valve 100 is radially compressed. For example, the base portion 152 can made of a relatively elastic material, such as TPU, that can stretch axially when the frame 102 is radially compressed. In some examples, the base portion 152 can be made of a fabric having yarns that are oriented generally parallel to the struts 116 of the frame 102, which allows the base portion 152 to elongate when the frame 102 is radially compressed. In some examples, the base portion 152 can comprise a composite including different materials, such a fabric embedded in or attached to an elastomeric layer (for example, a TPU layer).

### Delivery Apparatus

FIG. 7 shows a delivery apparatus 200, according to one example, that can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic heart valve into a heart.

The delivery apparatus 200 in the illustrated example of FIG. 7 is a balloon catheter comprising a handle 202 and a steerable, outer shaft 904 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 902 coaxially through the intermediate shaft 206 and the outer shaft 204 and proximally from the handle 202 coaxially through the intermediate shaft 206.

The outer shaft 904 and the intermediate shaft 206 can be configured to translate (for example, move) longitudinally, along a central longitudinal axis 220 of the delivery apparatus 200, relative to one another to facilitate delivery and positioning of a prosthetic heart valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 904.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 7), or to an alternate component at the distal end of the delivery apparatus 900 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 7, a prosthetic heart valve 250 (which can be one of the prosthetic heart valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic heart valve 250 is mounted in the radially compressed state on the valve mounting portion 224 (as shown in FIG. 7) and during delivery of the prosthetic heart valve to the target implantation site. As such, the distal tip portion 228 can be configured to resist movement of the prosthetic heart valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic heart valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Publication No. WO 2022/046585, which is incorporated by reference herein.

### Delivery Techniques

For implanting a prosthetic heart valve within the native aortic valve via a transfemoral delivery approach, the prosthetic heart valve is mounted in a radially compressed state along the distal end portion of a delivery apparatus. The prosthetic heart valve and the distal end portion of the delivery apparatus are inserted into a femoral artery and are advanced into and through the descending aorta, around the aortic arch, and through the ascending aorta. The prosthetic heart valve is positioned within the native aortic valve and radially expanded (for example, by inflating a balloon, actuating one or more actuators of the delivery apparatus, or deploying the prosthetic heart valve from a sheath to allow the prosthetic heart valve to self-expand). Alternatively, a prosthetic heart valve can be implanted within the native aortic valve in a transapical procedure, whereby the prosthetic heart valve (on the distal end portion of the delivery apparatus) is introduced into the left ventricle through a surgical opening in the chest and the apex of the heart and the prosthetic heart valve is positioned within the native aortic valve. Alternatively, in a transaortic procedure, a prosthetic heart valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic heart valve within the native mitral valve via a transseptal delivery approach, the prosthetic heart valve is mounted in a radially compressed state along the distal end portion of a delivery apparatus. The prosthetic heart valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic heart valve can be implanted within the native mitral valve in a transapical procedure, whereby the prosthetic heart valve (on the distal end portion of the delivery apparatus) is introduced into the left ventricle through a surgical opening in the chest and the apex of the heart and the prosthetic heart valve is positioned within the native mitral valve.

For implanting a prosthetic heart valve within the native tricuspid valve, the prosthetic heart valve is mounted in a radially compressed state along the distal end portion of a delivery apparatus. The prosthetic heart valve and the distal end portion of the delivery apparatus are inserted into a femoral vein and are advanced into and through the inferior vena cava, and into the right atrium, and the prosthetic heart valve is positioned within the native tricuspid valve. A similar approach can be used for implanting the prosthetic heart valve within the native pulmonary valve or the pulmonary artery, except that the prosthetic heart valve is advanced through the native tricuspid valve into the right ventricle and toward the pulmonary valve/pulmonary artery.

Another delivery approach is a transatrial approach whereby a prosthetic heart valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic heart valve (on the distal end portion of the delivery apparatus) is inserted through an incision in the chest and an incision made through the wall of the right ventricle (typically at or near the base of the heart) for implanting the prosthetic heart valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic heart valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame comprising a plurality of rows of angled struts arranged to form a plurality of rows of cells defining openings in the frame, wherein the frame is radially expandable and compressible between a radially expanded state and a radially compressed state; a valve structure positioned within the frame and configured to regulate the flow of blood through the frame in one direction; and an outer sealing member disposed on an outer surface of the frame, comprising: a base portion extending around the outer surface of the frame and covering the openings of at least one of the rows of cells; and at least one angled portion coupled to the base portion at an inflow end portion thereof and extending radially away from the base portion in a direction from the inflow end to an outflow end portion of the angled portion.

Example 2. The prosthetic heart valve of any example herein, particularly example 1, wherein the base portion and the angled portion are made of fabric.

Example 3. The prosthetic heart valve of any example herein, particularly any one of examples 1-2, wherein the prosthetic heart valve is devoid of an internal sealing member inside of the frame.

Example 4. The prosthetic heart valve of any example herein, particularly any one of examples 1-3, wherein the angled portion is completely unattached to the base portion along an entirety of the outflow end portion of the angled portion.

Example 5. The prosthetic heart valve of any example herein, particularly any one of examples 1-4, wherein the base portion is cylindrical.

Example 6. The prosthetic heart valve of any example herein, particularly any one of examples 1-5, wherein the base portion conforms to the outer surface of the frame when the frame is in the radially expanded state.

Example 7. The prosthetic heart valve of any example herein, particularly any one of examples 1-6, wherein the base portion is tensioned circumferentially when the frame is in the radially expanded state.

Example 8. The prosthetic heart valve of any example herein, particularly any one of examples 1-7, wherein the outer sealing member is sutured to selected struts of the frame.

Example 9. The prosthetic heart valve of any example herein, particularly any one of examples 1-8, wherein an outflow end portion of the base portion is folded radially outwards to form a folded portion.

Example 10. The prosthetic heart valve of any example herein, particularly any one of examples 1-9, wherein the row of cells covered by the base portion are at an inflow end of the frame.

Example 11. The prosthetic heart valve of any example herein, particularly any one of examples 1-10, wherein the angled portion assumes a frustoconical shape when the frame is in the radially expanded state.

Example 12. The prosthetic heart valve of any example herein, particularly any one of examples 1-11, wherein the angled portion tapers from the outflow end portion to the inflow end portion of the angled portion when the frame is in the radially expanded state.

Example 13. The prosthetic heart valve of any example herein, particularly any one of examples 1-12, wherein the base portion and the angled portion form an angle of approximately 40 degrees when the frame is in the radially expanded state.

Example 14. The prosthetic heart valve of any example herein, particularly any one of examples 1-13, wherein the angled portion can be placed in a flattened configuration when the frame is in the radially compressed state, such that the angled portion is flattened against an outer surface of the base portion.

Example 15. The prosthetic heart valve of any example herein, particularly example 14, wherein the angled portion is configured to self-expand from the flattened configuration to a frustoconical shape, wherein the angled portion is shape set to assume the frustoconical shape when the frame is in the radially expanded state.

Example 16. The prosthetic heart valve of any example herein, particularly any one of examples 1-15, wherein the at least one angled portion comprises a plurality of angled portions positioned along a height of the base portion.

Example 17. The prosthetic heart valve of any example herein, particularly example 16, wherein at least one of the plurality of angled portions at least partially overlaps an adjacent angled portion in an axial direction of the prosthetic heart valve.

Example 18. The prosthetic heart valve of any example herein, particularly any one of example 16, wherein none of the angled portions overlaps an adjacent angled portion in an axial direction of the prosthetic heart valve.

Example 19. A prosthetic heart valve comprising: a frame, wherein the frame is radially expandable and compressible between a radially expanded state and a radially compressed state; a valve structure positioned within the frame and configured to regulate the flow of blood through the frame in one direction; and an outer sealing member coupled to an outer surface of the frame, the outer sealing member comprising: a base portion circumferentially disposed around the outer surface of the frame; and a plurality of annular flap portions positioned along a height of the base portion, wherein each flap portion has an inflow end portion connected to the base portion and an outflow end portion unconnected to the base portion to define a pocket between the flap portion and the base portion that can receive retrograde blood.

Example 20. The prosthetic heart valve of any example herein, particularly any one of example 19, wherein the inflow end portion of each flap portion has a diameter that is smaller than a diameter of the outflow end portion of the flap portion.

Example 21. The prosthetic heart valve of any example herein, particularly any one of examples 19-20, wherein the prosthetic heart valve is devoid of an internal sealing member inside of the frame.

Example 22. The prosthetic heart valve of any example herein, particularly any one of examples 19-21, wherein each flap portion is completely unattached to the base portion along an entirety of the outflow end portion of the flap portion.

Example 23. The prosthetic heart valve of any example herein, particularly any one of examples 19-22, wherein the base portion is cylindrical.

Example 24. The prosthetic heart valve of any example herein, particularly any one of examples 19-23, wherein the base portion conforms to the outer surface of the frame when the frame is in a radially expanded state.

Example 25. The prosthetic heart valve of any example herein, particularly any one of examples 19-24, wherein the base portion is tensioned circumferentially when the frame is in the radially expanded state.

Example 26. The prosthetic heart valve of any example herein, particularly any one of examples 19-25, wherein the base portion can elongate axially when the frame is radially compressed to the radially compressed state.

Example 27. The prosthetic heart valve of any example herein, particularly any one of examples 19-26, wherein the base portion has an outflow end region that is devoid of any flap portions.

Example 28. The prosthetic heart valve of any example herein, particularly any one of examples 19-27, wherein each of the flap portions have a frustoconical shape when the frame is in the radially expanded state.

Example 29. The prosthetic heart valve of any example herein, particularly example 28, wherein the flap portions are shape set to assume the frustoconical shape when the prosthetic heart valve is in the radially expanded state.

Example 30. The prosthetic heart valve of any example herein, particularly any one of examples 19-29, wherein the frame comprises a plurality of rows of angled struts arranged to form a plurality of rows of cells defining openings in the frame and the base portion covers all of the openings of at least one of the rows of the cells.

Example 31. The prosthetic heart valve of any example herein, particularly example 30, wherein the row of cells covered by the base portion are at an inflow end of the frame.

Example 32. The prosthetic heart valve of any example herein, particularly example 31, wherein the valve structure comprises a plurality of leaflets forming a plurality of commissures connected to the frame, wherein each leaflet has a cusp edge portion, wherein adjacent cusp edge portions of adjacent leaflets are spaced from each other to define gaps between adjacent leaflets upstream of the commissures, and wherein the base portion extends axially along the frame from the inflow end of the frame to a location upstream of the gaps such that the openings in the frame where the gaps are located are covered by the base portion.

Example 33. The prosthetic heart valve of any example herein, particularly example 32, wherein a portion of the frame downstream of the commissures is uncovered by the base portion.

Example 34. The prosthetic heart valve of any example herein, particularly any one of examples 19-33, wherein at least two of the flap portions partially overlap each other in an axial direction of the prosthetic heart valve.

Example 35. The prosthetic heart valve of any example herein, particularly any one of examples 19-33, wherein none of flap portions overlaps an adjacent flap portion in an axial direction of the prosthetic heart valve.

Example 36. The prosthetic heart valve of any example herein, particularly any one of examples 19-35, wherein the inflow end portion of each flap portion is sutured to the base portion.

Example 37. A prosthetic heart valve comprising: a frame, wherein the frame is radially expandable and compressible between a radially expanded state and a radially compressed state; a valve structure positioned within the frame and configured to regulate the flow of blood through the frame in one direction; an outer sealing member disposed on an outer surface of the frame, the outer sealing member comprising: a base portion coupled to the outer surface of the frame and covering openings in the frame to prevent blood outside of the prosthetic valve from flowing into the frame via the openings; and at least one angled portion, wherein the angled portion comprises: an inflow end portion continuously coupled to the base portion along an entirety of the inflow end portion; and an outflow end portion opposite the inflow end portion, wherein the outflow end portion is completely unattached to the base portion.

Example 38. The prosthetic heart valve of any example herein, particularly example 37, wherein the base portion and the angled portion are made of fabric.

Example 39. The prosthetic heart valve of any example herein, particularly any one of examples 37-38, wherein the prosthetic heart valve is devoid of an internal sealing member inside of the frame.

Example 40. The prosthetic heart valve of any example herein, particularly any one of examples 37-39, wherein the angled portion is coupled to the base portion using one of mechanical fasteners, sutures, adhesives, and ultrasonic welds.

Example 41. The prosthetic heart valve of any example herein, particularly any one of examples 37-40, wherein the base portion is cylindrical.

Example 42. The prosthetic heart valve of any example herein, particularly any one of examples 37-41, wherein the base portion conforms to the outer surface of the frame when the frame is in the radially expanded state.

Example 43. The prosthetic heart valve of any example herein, particularly any one of examples 37-42, wherein the base portion is tensioned circumferentially when the frame is in the radially expanded state.

Example 44. The prosthetic heart valve of any example herein, particularly any one of examples 37-43, wherein an outflow end portion of the base portion is folded radially outwards to form a folded portion.

Example 45. The prosthetic heart valve of any example herein, particularly any one of examples 37-43, wherein the row of cells covered by the base portion are at an inflow end of the frame.

Example 46. The prosthetic heart valve of any example herein, particularly any one of examples 37-45, wherein the angled portion assumes a frustoconical shape when the frame is in the radially expanded state.

Example 47. The prosthetic heart valve of any example herein, particularly any one of examples 37-46, wherein the angled portion tapers from the outflow end portion to the inflow end portion of the angled portion when the frame is in the radially expanded state.

Example 48. The prosthetic heart valve of any example herein, particularly any one of examples 37-47, wherein the angled portion is configured to be placed in a flattened configuration when the frame is in the radially compressed state, such that the angled portion is flattened against an outer surface of the base portion.

Example 49. The prosthetic heart valve of any example herein, particularly example 48, wherein the angled portion is configured to self-expand from the flattened configuration to a frustoconical shape, wherein the angled portion is shape set to assume the frustoconical shape when the frame is in the radially expanded state.

Example 50. The prosthetic heart valve any example herein, particularly any one of examples 37-49, wherein the prosthetic heart valve is devoid of any material on an inner surface of the frame that occludes the cells of the frame.

Example 51. The prosthetic heart valve of any example herein, particularly any one of examples 37-50, wherein at least 90% of an inner surface of the frame is not covered by any material.

Example 52. The prosthetic heart valve any example herein, particularly any one of examples 37-51, wherein the at least one angled portion comprises a plurality of angled portions positioned along a height of the base portion.

Example 53. The prosthetic heart valve any example herein, particularly example 52, wherein at least two of the plurality of angled portions partially overlap an adjacent angled portion in an axial direction of the prosthetic heart valve.

Example 54. The prosthetic heart valve any example herein, particularly example 52, wherein the plurality of angled portions do not overlap in an axial direction of the prosthetic heart valve.

Example 55. A prosthetic heart valve of any example herein, particularly any one of examples 1-54, wherein the prosthetic heart valve is sterilized.

The features described herein with regard to any example can be combined with other features described in any one or more of the other examples, unless otherwise stated. For example, any one or more of the features of one outer sealing member can be combined with any one or more features of another outer sealing member. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame comprising a plurality of rows of angled struts arranged to form a plurality of rows of cells defining openings in the frame, wherein the frame is radially expandable and compressible between a radially expanded state and a radially compressed state;
a valve structure positioned within the frame and configured to regulate the flow of blood through the frame in one direction; and
an outer sealing member disposed around an outer surface of the frame, comprising:
a base portion extending around the outer surface of the frame and covering the openings of at least one of the rows of cells; and
at least one angled portion coupled to the base portion at an inflow end portion thereof and extending radially away from the base portion in a direction from the inflow end to an outflow end portion of the angled portion.

2. The prosthetic heart valve of claim 1, wherein the prosthetic heart valve is devoid of an internal sealing member inside of the frame.

3. The prosthetic heart valve of claim 1, wherein the angled portion is completely unattached to the base portion along an entirety of the outflow end portion of the angled portion.

4. The prosthetic heart valve of claim 1, wherein the base portion conforms to the outer surface of the frame when the frame is in the radially expanded state.

5. The prosthetic heart valve of claim 1, wherein an outflow end portion of the base portion is folded radially outwards to form a folded portion.

6. The prosthetic heart valve of claim 1, wherein the angled portion assumes a frustoconical shape when the frame is in the radially expanded state.

7. The prosthetic heart valve of claim 1, wherein the angled portion tapers from the outflow end portion to the inflow end portion of the angled portion when the frame is in the radially expanded state.

8. The prosthetic heart valve of claim 1, wherein the angled portion can be placed in a flattened configuration when the frame is in the radially compressed state, such that the angled portion is flattened against an outer surface of the base portion.

9. The prosthetic heart valve of claim 8, wherein the angled portion is configured to self-expand from the flattened configuration to a frustoconical shape, wherein the angled portion is shape set to assume the frustoconical shape when the frame is in the radially expanded state.

10. The prosthetic heart valve of claim 1, wherein the at least one angled portion comprises a plurality of angled portions positioned along a height of the base portion.

11. The prosthetic heart valve of claim 10, wherein at least one of the plurality of angled portions at least partially overlaps an adjacent angled portion in an axial direction of the prosthetic heart valve.

12. The prosthetic heart valve of claim 10, wherein none of the angled portions overlaps an adjacent angled portion in an axial direction of the prosthetic heart valve.

13. A prosthetic heart valve comprising:
a frame, wherein the frame is radially expandable and compressible between a radially expanded state and a radially compressed state;
a valve structure positioned within the frame and configured to regulate the flow of blood through the frame in one direction; and
an outer sealing member coupled to an outer surface of the frame, the outer sealing member comprising:
a base portion circumferentially disposed around the outer surface of the frame; and
a plurality of annular flap portions positioned along a height of the base portion, wherein each flap portion has an inflow end portion connected to the base portion and an outflow end portion unconnected to the base portion to define a pocket between the flap portion and the base portion that can receive retrograde blood.

14. The prosthetic heart valve of claim 13, wherein the inflow end portion of each flap portion has a diameter that is smaller than a diameter of the outflow end portion of the flap portion.

15. The prosthetic heart valve of claim 13, wherein the flap portions are shape set to assume a frustoconical shape when the prosthetic heart valve is in the radially expanded state.

16. The prosthetic heart valve of claim 13, wherein the frame comprises a plurality of rows of angled struts arranged to form a plurality of rows of cells defining openings in the frame and the base portion covers all of the openings of at least one of the rows of the cells.

17. The prosthetic heart valve of claim 16, wherein the row of cells covered by the base portion are at an inflow end of the frame.

18. The prosthetic heart valve of claim 17, wherein the valve structure comprises a plurality of leaflets forming a plurality of commissures connected to the frame, wherein each leaflet has a cusp edge portion, wherein adjacent cusp edge portions of adjacent leaflets are spaced from each other to define gaps between adjacent leaflets upstream of the commissures, and wherein the base portion extends axially along the frame from the inflow end of the frame to a location upstream of the gaps such that the openings in the frame where the gaps are located are covered by the base portion.

19. The prosthetic heart valve of claim 18, wherein a portion of the frame downstream of the commissures is uncovered by the base portion.

20. A prosthetic heart valve comprising:
a frame, wherein the frame is radially expandable and compressible between a radially expanded state and a radially compressed state;
a valve structure positioned within the frame and configured to regulate the flow of blood through the frame in one direction;
an outer sealing member disposed on an outer surface of the frame, the outer sealing member comprising:
a base portion coupled to the outer surface of the frame and covering openings in the frame to prevent blood outside of the prosthetic heart valve from flowing into the frame via the openings; and
at least one angled portion, wherein the angled portion comprises:
an inflow end portion continuously coupled to the base portion along an entirety of the inflow end portion; and
an outflow end portion opposite the inflow end portion,
wherein the outflow end portion is completely unattached to the base portion.
